(19) United States
(12) Patent Application Publication
(10) Pub. No.: US 2026/0033562 A1
(43) Pub. Date: Feb. 5, 2026

(54) MOISTURE MANAGEMENT OUTER WEAR GARMENTS

(71) Applicant: MODIBODI AUSTRALIA PTY LTD, New South Wales (AU)

(72) Inventors: Kristy CHONG, New South Wales (AU); Alice WARNER, New South Wales (AU)

(73) Assignee: MODIBODI AUSTRALIA PTY LTD, New South Wales (AU)

(21) Appl. No.: 19/099,376

(22) PCT Filed: Jul. 29, 2022
(86) PCT No.: PCT/AU2022/050817

§ 371 Date: Jan. 28, 2025

(51) Int. Cl. A41D 1/08; A41D 13/00; A41D 31/12

(52) U.S. Cl. CPC A41D 1/088; A41D 13/0017; A41D 31/125

(57) ABSTRACT

Described herein is a launderable moisture management outer wear pant garment for exercise comprising: a moisture management fabric assembly for managing bodily fluid secretions disposed in an internal crotch region of the pant garment so as to closely contact a wearer's skin during use; wherein the moisture management fabric assembly is at least partially attached to a fabric saddle which is fixedly attached to an interior surface of the pant garment along a part or full length of a fabric saddle boundary which is distally located from the crotch region; wherein the fabric saddle and the distal location of the fabric saddle boundary indirectly attaches the moisture management fabric assembly to the outer wear pant garment in a manner that avoids externally visible seams in the crotch region.

### TECHNICAL FIELD

This invention relates to launderable outer wear garments suitable for use during exercise. More particularly, this invention relates to outer wear pant garments for exercise that incorporate a discreetly mounted fabric assembly for managing bodily fluid secretions such as period blood and/or discharge, urine, sweat, etc.

### BACKGROUND

Femtech products including reusable/launderable moisture management garments such as incontinence or period underwear are increasingly popular. Such products are an improvement over tampons, pads and related disposable sanitary articles in terms of sustainability and eco friendliness. However, for certain outer wear garments—such as sports and/or form fitting garments—the layering of incontinence or period underwear under the outer wear garment may be uncomfortable, bulky and/or indiscreet with the presence of a visible panty line (VPL). It would therefore be desirable to have sustainable and eco-friendly alternatives to layering incontinence or period underwear underneath pant outer wear.

Past attempts to provide alternatives to underpant/pant layering include sewing moisture management panels into the crotch region of exercise tights. Although this solution overcomes issues caused by layering, such as added heat retention, added bulk, and the potential for shifting or movement of the outer wear over the underwear, such garments have the drawback that typically the moisture management panels, and particularly their points of attachment to the pant garment, are externally visible. This solution is thus unsuitable for persons wishing for their need or preference for moisture management capability to remain discreet. There is also the possibility of the misunderstanding that the outer wear pants are underwear. It would therefore be desirable to provide alternative pant garments capable of managing bodily fluid secretions without outwardly obvious/externally visible signs of the moisture management function in the crotch region.

Improved moisture management pant garments that address one or more of the above problems or at least provide a useful alternative are thus desirable. In one aspect, the invention solves the problem of hiding a moisture management fabric assembly discretely in a close-fitting, figure-hugging pant garment such as exercise pants, leggings or tights, while avoiding a hint or suggestion of the presence of the moisture management fabric assembly though avoiding a VPL and/or externally visible seams/stitching in the crotch region of the pant garment.

A reference herein to a patent document or other matter which is given as prior art is not to be taken as an admission that the document or matter was known or that the information it contains was part of the common general knowledge as at the priority date of any of the claims.

### SUMMARY OF INVENTION

Where the terms “comprise”, “comprises” and “comprising” are used in the specification (including the claims) they are to be interpreted as specifying the stated features, integers, steps or components, but not precluding the presence of one or more other features, integers, steps or components, or group thereof.

According to a first aspect of the present invention, there is provided a launderable moisture management outer wear exercise pant garment comprising:
a moisture management fabric assembly for managing bodily fluid secretions, disposed in an internal crotch region of the pant garment so as to closely contact a wearer's skin during use;
wherein the moisture management fabric assembly is at least partially attached to a fabric saddle which is fixedly attached to an interior surface of the pant garment along a part or full length of a fabric saddle boundary which is distally located from the crotch region,
wherein the fabric saddle and the distal location of the fabric saddle boundary indirectly attaches the moisture management fabric assembly to the outer wear pant garment in a manner that avoids externally visible seams in the crotch region.

In the garment described herein, no portion of the moisture management fabric assembly may be directly attached to the crotch region of the pant garment.

The fabric saddle may be fixedly attached to the interior surface of the pant garment solely along a part or full length of the fabric saddle boundary and remain unattached to the interior surface of the pant garment along all other surfaces of the fabric saddle. The fabric saddle may be fixedly attached to the interior surface of the pant garment solely along a full length of the fabric saddle boundary and remains unattached to the interior surface of the pant garment along all other surfaces of the fabric saddle. The fabric saddle may have extremities that extend in length from at least two edges of the moisture management fabric assembly to the fabric saddle boundary. The fabric saddle may have extremities that extend in length to the fabric saddle boundary. In one embodiment, the fabric saddle may have extremities that extend in length from at least four, or optionally all, edges of the moisture management fabric assembly to the fabric saddle boundary. The moisture management fabric assembly may be located over a central saddle point of the fabric saddle. The fabric saddle may flare upwardly towards a torso opening of the pant garment such that the fabric saddle boundary is partially situated in at least a front abdominal region and at least a lower back region of the torso opening. The fabric saddle may have extremities that extend in length and in opposite directions from at least two edges of the moisture management fabric assembly to the fabric saddle boundary, wherein the extremities of the fabric saddle flare upwardly towards a torso opening of the pant garment such that the fabric saddle boundary is partially situated in at least a front abdominal region and at least a lower back region of the torso opening. The fabric saddle may have a generally flared hyperbolic paraboloid shape comprising a central saddle point disposed in the crotch region of the pant garment. The fabric saddle may extend internally into each leg opening of the pant garment such that at portion of the fabric saddle boundary is partially situated in each of leg opening. At least a portion of the fabric saddle boundary may be located proximate to a general knee area of the pant garment and/or at least a portion of the fabric saddle boundary may be located proximate to a general lower thigh area of the pant garment. The fabric saddle may comprise two or more fabric pieces connected together. The fabric of the fabric saddle may comprise one or more layers of a fabric, preferably, at least one of which is a fabric mesh. The fabric of the fabric saddle may comprise one or more layers of a natural fabric, a synthetic fabric, a semi synthetic fabric or a combination thereof. The fabric of the fabric saddle may comprise a synthetic fabric, preferably a nylon/spandex blend.

The moisture management fabric assembly may have opposing long edges situated between a wearer's legs proximate each leg opening of the pant garment and opposing short edges, wherein the moisture management fabric assembly is fixedly attached to the fabric saddle at least along the opposing long edges. The moisture management fabric assembly may comprise a fluid absorbing fabric core for trapping bodily fluid secretions. The moisture management fabric assembly may comprise a fluid absorbing fabric core including one or more layers of an absorbent, quick drying fabric, preferably an absorbent, quick drying microfibre fabric such as a polyester microfibre fabric, a polyamide microfibre fabric or polyamide blend microfibre fabric such as a polyamide/elastane blend fabric, or a polyester fabric, or polyester blend microfibre fabric such as a polyester/polyamide blend microfibre fabric; or an absorbent, quick drying fabric manufactured from a cellulosic material, preferably cotton or a cotton blend such as a cotton and polyester blend, Tencel, hemp or bamboo, optionally wherein the cellulosic material is treated to improve absorbency (e.g., bamboo charcoal); or a natural fabric such as wool, especially Merino wool, most preferably a polyester/polyamide blend microfibre fabric. The moisture management fabric assembly may comprise a fluid wicking fabric for directly contacting a wearer's skin in use, wherein the fluid wicking fabric is positioned adjacent to a fluid absorbing fabric core. The moisture management fabric assembly may comprise a fluid wicking fabric selected from cotton, a cotton blend such as a cotton elastane jersey, Modal, polyester or polyester blends such as polyester elastane, bamboo fleece, wool such as Merino wool, or a brushed fabric having a brushed or otherwise textured surface having fibres displaced from the fabric knit, such as a brushed wool, particularly brushed Merino wool, a brushed wicking polyester, a brushed wicking polypropylene, a brushed wicking nylon, a brushed wicking micromodal, a brushed wicking bamboo, a brushed cotton, or a composite or blend thereof, most preferably brushed Merino wool. The moisture management fabric assembly may comprise a fluid barrier fabric to prevent fluid leaking outside the pant during use, wherein the fluid barrier fabric is positioned adjacent to the fluid absorbing fabric core, preferably wherein the fluid barrier fabric comprises a fluid proof fabric, a fluid resistant fabric or a fluid repellent fabric, optionally further provided with a fluid proof, fluid resistant or fluid repellent polymer film such a polyurethane film or a thermoplastic polyurethane film. In some embodiments, the fluid barrier fabric may dispensed with and a fluid proof, fluid resistant or fluid repellent polymer film or web can be used instead, e.g., bonded to the outer face of the fluid absorbing fabric core component. The externally visible seams in the crotch region of the pant garment may exclude seams of a crotch shaping panel in an outer layer or shell of the pant garment, if present. The garment may be in the form of tights, leggings or shorts, including cycle shorts.

According to a second aspect of the present invention, there is provided use of a fabric saddle for positioning a moisture management fabric assembly in an internal crotch region of a launderable moisture management outer wear exercise pant garment, said positioning being closely in contact with a wearer's skin during use for managing bodily fluid secretions,
wherein the fabric saddle is fixedly attached to an interior surface of the pant garment along a part or full length of a fabric saddle boundary which is distally located from the crotch region and the moisture management fabric assembly is at least partially attached to the fabric saddle; and
wherein the fabric saddle and the distal location of the fabric saddle boundary indirectly attaches the moisture management fabric assembly to the outer wear pant garment in a manner that avoids externally visible seams in the crotch region.

### BRIEF DESCRIPTION OF DRAWINGS

Embodiments of the invention will herein be illustrated by way of example only with reference to the accompanying drawings in which:

FIG. 1 illustrates a non-limiting example of a generally flared hyperboloid paraboloid shaped fabric saddle of one aspect of the invention.

FIG. 2 (a) illustrates a front external view of one embodiment of a pant garment as described herein in the form of leggings or tights; and (b) illustrates a front view of the same pant garment as FIG. 2 (a) but when viewed inside out.

FIG. 3 (b) illustrates a rear external view of one embodiment of a pant garment as described herein in the form of leggings or tights; and (a) illustrates a rear view of the same pant garment as FIG. 3 (b) but when viewed inside out.

FIG. 4 (b) illustrates a side on external view of one embodiment of a pant garment as described herein in the form of leggings or tights; and (a) illustrates a side on view of the same pant garment FIG. 4 (b) but when viewed inside out.

FIG. 5 (b) illustrates a side on inner leg/walking external view of one embodiment of a pant garment as described herein in the form of leggings or tights; and (a) a side on inner leg/walking view of the same pant garment as FIG. 5 (b) but when viewed inside out.

FIG. 6 (b) illustrates a front external view of one embodiment of a pant garment as described herein in the form of leggings or tights; and (a) illustrates a front view of the same pant garment as FIG. 6 (b) but when viewed inside out.

FIG. 7 (b) illustrates a back external view of one embodiment of a pant garment as described herein in the form of leggings or tights; and (a) illustrates a back view of the same pant garment as FIG. 7 (b) but when viewed inside out.

FIG. 8 (b) illustrates a side on external view of another embodiment of a pant garment as described herein in the form of leggings or tights; and (a) illustrates a side on view of the same pant garment FIG. 8 (b) but when viewed inside out.

FIG. 9 (a) illustrates a front external view of one embodiment of a pant garment as described herein in the form of cycle shorts; and (b) illustrates a front view of the same pant garment as FIG. 9 (a) but when viewed inside out.

10 FIG. 10 (a) illustrates a rear external view of one embodiment of a pant garment as described herein in the form of cycle shorts; and (b) illustrates a rear view of the same pant garment as FIG. 10 (a) but when viewed inside out.

FIG. 11 illustrates a side on external view of one embodiment of a pant garment as described herein in the form of cycle shorts.

FIG. 12 illustrates, on a stylised human body, a non-limiting example of (a) a front view of a crotch region; (b) a back view of a crotch region; (c) a top view of a crotch region as defined according to one embodiment herein as viewed through the torso opening of a pant garment; and (d) a top view of a pant garment according to one embodiment herein.

FIG. 13 (a) illustrates a front internal (inside out) view and (b) illustrates a rear internal (inside out) view of one embodiment of a pant garment as described herein in the form of cycle shorts and shows the fabric saddle boundary.

FIG. 14 (a) illustrates a front internal (inside out) view and (b) illustrates a top internal view of one embodiment of a pant garment as described herein.

### DETAILED DESCRIPTION

Described herein is a launderable (preferably machine washable) moisture management outer wear pant garment for exercise comprising a moisture management fabric assembly (MMFA) for managing bodily fluid secretions. The MMFA is disposed in an internal crotch region of the pant garment so as to closely contact a wearer's skin during use, where it can efficiently wick moisture away from the wear's skin where the MMFA is in skin contact. The moisture management fabric assembly is at least partially attached to a fabric saddle which is itself fixedly attached to an interior surface of the pant garment along a part or full length of a fabric saddle boundary which is distally located from the crotch region. There is no direct attachment of the MMFA to the crotch of the pant. That is, there are no parts of the MMFA directly connected or attached to the crotch of the pant, through stitching or otherwise. Instead, the fabric saddle and the distal location of the fabric saddle boundary indirectly attaches the moisture management fabric assembly to the outer wear pant garment in a type of surrogate attachment manner that advantageously avoids externally visible seams in the crotch region of the pant. The structure and arrangement described here conveniently solves the problem of hiding a moisture management fabric assembly discretely in a close-fitting, figure-hugging pant garment such as exercise pants, leggings or tights while avoiding a hint or suggestion of the presence of the moisture management fabric assembly though avoiding a VPL and/or externally visible seams/stitching in the crotch region of the pant garment.

The pant garment described herein advantageously avoids externally visible seams in the crotch region that may associate the pant in the consumer's mind with underwear, whilst still offering high levels of moisture management capability. Though the structure described herein, this can be achieved for figure hugging exercise pants such as leggings or tights, due to their nature are aesthetically unforgiving. The inventors believe that this is the first time a fabric saddle has been used for mounting a moisture management fabric assembly discretely in a figure-hugging pant garment such as leggings or tights. The fabric saddle described herein offers several advantages over prior garments. By being fixedly attached to the inside of the pant garment at a fabric saddle boundary distally located from the crotch region, the fabric saddle described herein assists to positionally secure the MMFA to the pant garment without need for seams in the crotch region that may otherwise draw attention to the presence of the fabric assembly. To further explain, the fabric saddle is fixedly attached to the pant garment, in some embodiments by intentionally visible stitching means which are intentionally positioned in a location well away from the crotch region, thereby drawing visual attention away from the crotch region at the same time as avoiding stitching of the MMFA in the crotch region. Indeed, the clever placement of intentionally visible stitching means used in the pant of the invention provides enhanced aesthetic qualities to the pant garment in addition to the functionality described herein. Another advantage of the fabric saddle in some embodiments is that the fabric saddle itself assists in disguising the presence and/or shape of the MMFA by providing an additional layer of fabric between the MMFA and the shell or exterior fabric of the pant garment.

This additional layer assists in softening or otherwise diminishing the visible outline of the MMFA. Utilising a fabric saddle as described herein is particularly advantageous for form-fitting garments such as leggings, tights, and cycle shorts, as these garments are typically designed to hug a wearer's figure, including in the crotch region, for ease and freedom of movement as well as support. Form-fitting garments that hug a wearer's figure, and in particular are tight in the crotch region, are more likely to externally reveal the presence and shape of underwear and/or moisture management pads being worn under the garment.

As used herein, the term “crotch region” of the pant garment described herein shall be understood as including the crotch, which is the part of the human body between the legs where they join the torso, and extending from a base in the crotch to the top of the mons pubis at the front (ventral face) and to the anus as wide as the sit bones at the back (dorsal face). In other words, the term “crotch region” shall be understood as including the mons pubis on the ventral face, the crotch, and an area extending on the dorsal face from the crotch to as wide as the sit bones at anus level. In some embodiments, the crotch region extends down the ventral face of the pant garment over the mons pubis, through the crotch, and gradually widens up the dorsal face of the buttocks to the width of the sit bones at anus level. In some embodiments, the crotch region extends down the ventral face of the pant garment in a generally inverted trapezoidal shape over the mons pubis, generally rectangularly through the crotch, and gradually widens up the dorsal face (buttocks) in a generally trapezoidal shape to the width of the sit bones at anus level. Anatomically, in some embodiments, this may correspond to a generally flared rectangle having a generally elongate shape that flares outwardly (i.e., away) from its longitudinal axis at each end, and that is bounded, with respect to a standing person wearing the pant garment (the wearer), by an imaginary horizontal line at the front that passes across the top of the mons pubis over the public symphysis joint of the wearer and extends in a generally inverted triangular shape down over the mons pubis to between a wearer's legs in the creases formed at the joins between each inner leg and the wearer's torso, and extends upwardly at the rear to an imaginary line passing across a wearer's buttocks at anus level to a width corresponding to the distance from the base of the left ischium (ischial tuberosity) to the base of the right ischium (ischial tuberosity) (i.e., across a wearer's buttocks from one sit bone to the other). It will be understood that the “internal crotch region” refers to this region as it sits on the interior or skin-facing surface of the pant garment when worn by a wearer.

As used herein, the term “distally located from the crotch region” may refer, in some embodiments, to the fabric saddle boundary being fixedly attached to an interior surface of the pant garment at a distance of at least 1 cm from each boundary of the crotch region as defined herein, or in other embodiments, to the fabric saddle boundary being fixedly attached to an interior surface of the pant garment at a distance of at least 2 cm, at least 3 cm, at least 4 cm, at least 5 cm, at least 6 cm, at least 7 cm, at least 8 cm, at least 9 cm, at least 10 cm, at least 12 cm, at least 14 cm, or at least 16 cm from each boundary of the crotch region. In some embodiments, the fabric saddle boundary is fixedly attached to an interior surface of the pant garment at a distance of at least 2-3 cm from each boundary of the crotch region as defined herein. In some embodiments, the fabric saddle boundary is fixedly attached to an interior surface of the pant garment at a distance of at least 3-5 cm from each boundary of the crotch region as defined herein. In other words, the fabric saddle boundary is never fixedly attached to an interior surface of the pant garment closer than 2-3 cm, or 3-5 cm, from the crotch region as defined herein. The distance measurement in centimetres herein may be a linear measurement taken from any point along the crotch region boundary to any point along the fabric saddle boundary where it is fixedly attached to an interior surface of the pant garment (but is suitably taken from the point at which the fabric saddle boundary fixedly attached to the interior surface of the pant garment is closest to the crotch region). In some embodiments, the term “distally located from the crotch region” refers to the fabric saddle boundary being fixedly attached to an interior surface of the pant garment at a distance of between at least 1 cm and at least 10 cm from each boundary of the crotch region as defined herein, or of between at least 5 cm and at least 25 cm from each boundary, or of between at least 1 cm and at least 20 cm from each boundary, or of between at least 7 cm and at least 15 cm from each boundary, or of between at least 10 cm and at least 30 cm from each boundary, or of between at least 1 cm and at least 30 cm from each boundary of the crotch region as defined herein. In some embodiments, the term “distally located from the crotch region” refers to the fabric saddle boundary being fixedly attached to an interior surface of the pant garment at a distance of between at least 3 cm and 10 cm from each boundary of the crotch region as defined herein. It will be understood that the fabric saddle boundary need not be equidistant across its full distance from each boundary of the crotch region. Indeed, in some embodiments, the fabric saddle boundary is intentionally non-equidistantly distally located from the crotch region so as to allow shaping of the boundary to create aesthetically pleasing lines. In some embodiments, the term means a location that is in the leg opening. In some embodiments, the term means a location that is at a waistband of the pant garment, such as in a general abdominal region or a general lower back region. In some embodiments, the terms term “distally located from the crotch region” means any location in the leg opening that is closer to the knee than to the crotch area/region. In some embodiments, the term “distally located from the crotch region” means any location at the torso opening that is closer to the waistband than to the crotch area/region. In other embodiments, the term “distally located from the crotch region” means that the fabric saddle boundary is located outside the boundary of the MMFA. In some embodiments, the term means any location that is not in the area of the pubis and/or buttock when the pant garment is worn.

In one embodiment, no portion of the moisture management fabric assembly (MMFA) is directly attached to the inner (or outer) crotch region of the pant garment. This configuration may also be referred to herein as a “floating” moisture management fabric assembly (“floating” MMFA). In such an embodiment, the MMFA is separate from, detached from or “floats” in the pant garment by virtue of being partially attached to a fabric saddle. It will be understood that the fabric saddle itself is directly attached at its fabric saddle boundary to an inner part of the outer wear garment, in such a way that the MMFA (and the fabric saddle where the MMFA is at least partially attached to it) is at least separatable from contact with the interior crotch region of the pant garment. In some embodiments, the MMFA (and the fabric saddle where the MMFA is partially attached to it) “floats” or is separable from contact with the interior surface of the pant garment from the crotch region up to the fabric saddle boundary. The floating MMFA may advantageously allow for enhanced drying air contact with the MMFA by gently pulling the MMFA and the interior crotch portion of the pant garment away from each other while air drying. In some embodiments, the “floating” MMFA is freely separatable from contact with the interior surface of the pant garment and is also partially separatable from the fabric saddle. Such configuration may further enhance drying air contact with the MMFA and is described in greater detail elsewhere herein.

In one embodiment, the fabric saddle is fixedly attached to interior surfaces of the pant garment solely along a part or full length of the fabric saddle boundary and remains unattached to the interior surface of the pant garment along all other surfaces of the fabric saddle. The means of fixed attachment are not particularly limited, but may in some embodiments be machine stitching, circular knitting knitted stitching, bonding, welding, adhesive seam tape, or binding, or a combination of any two or more of these. Those of skill in the art will appreciate that circular knitting is a technique of knitting in a circular or tubular form and can create seamless fabric shapes by varying the tension or length of the stitches. Those of skill in the art will appreciate that heat sealed seams may be formed using adhesive seam tape, which is usually comprised of an adhesive layer and a barrier layer and which are often polyurethane based. Adhesive seam tape can be applied to a seam using any suitable, commercially available hot air taping machinery, where it can join fabric pieces together without the need for stitching/thread. Those of skill in the art will appreciate that welding refers to the thermal bonding and sealing of seams in knitted, woven, and non-woven thermoplastic materials such as nylon and polyester without adhesives, chemical binders, staples, needle, or thread. Heat, speed, and pressure of the welding strip can be adjusted to fuse the seam of two pieces of fabric. Bonding more generally utilises adhesive(s) in combination with heat, speed, and pressure to fuse two pieces of non-synthetic fabric together. In one embodiment, the fabric saddle is fixedly attached to the interior surface of the pant garment along an entire length of the fabric saddle boundary.

In one embodiment, the fabric saddle has extremities that extend in length to the fabric saddle boundary. For the avoidance of confusion, the fabric saddle boundary will be understood to refer to the complete, continuous edge of the fabric saddle. In some embodiments, the entire fabric saddle boundary is fixedly attached to the interior surface of the pant garment. In other embodiments, part of the fabric saddle boundary is fixedly attached to the interior surface of the pant garment.

The term “extremities” when referring to the fabric saddle encompasses parts of the fabric saddle that extend from the MMFA disposed in the internal crotch region to the fabric saddle boundary. In one embodiment, the fabric saddle has a generally flared hyperbolic paraboloid shape comprising a central saddle point disposed in the crotch region of the pant garment. A non-limiting example of a simple generally flared hyperboloid paraboloid shape is illustrated in FIG. 1. The term “generally flared” as used herein describes the tendency of curved portions of the paraboloid projecting above the central saddle point to flatten or have a steep gradient to accommodate or fit onto the abdominal and lower back/upper buttock areas of a wearer, and for the curved portions of the paraboloid projecting below the central saddle point to extend down each leg of a wearer, optionally wrapping partially or fully around each leg by means of an asymmetric extension of the paraboloid boundary.

In one embodiment, the fabric saddle flares upwardly towards a torso opening of the pant garment such that the fabric saddle boundary is partially situated in at least a front abdominal region and at least a lower back region of the torso opening. It will be understood that the exact location of the fabric saddle boundary on the front abdominal region and lower back region is not particularly limited. By way of non-limiting example only, the fabric saddle boundary may be partially situated above a waist seam defining a waist band, may be partially situated below a waist seam defining a waist band, or may extend to a terminal proximal boundary of the torso opening, or may be partially situated in any two or more of these locations. In one embodiment, the fabric saddle has extremities that extend in length and in opposite directions from the moisture management fabric assembly to the fabric saddle boundary, where the extremities of the fabric saddle flare upwardly towards a torso opening of the pant garment such that the fabric saddle boundary is partially situated in at least a front abdominal region and at least a lower back region of the torso opening.

In one embodiment, the fabric saddle has extremities that extend in length to the fabric saddle boundary and the extremities extend upwardly towards a torso opening of the pant garment to the fabric saddle boundary partially situated in at least a front abdominal region and at least a lower back region of the torso opening. In one embodiment, the MMFA is located over a central saddle point of the fabric saddle. In one embodiment, the central saddle point of the fabric saddle is located at or proximate to the crotch of a wearer.

In one embodiment, the fabric saddle extends internally into each leg opening of the pant garment such that a portion of the fabric saddle boundary is partially situated in each leg opening. In another embodiment, the fabric saddle has extremities that extend in length to the fabric saddle boundary and the extremities extend internally into each leg opening of the pant garment to a portion of the fabric saddle boundary partially situated in each leg opening.

In one embodiment, the MMFA is at least partially attached to the fabric saddle to enable physical separation of the MMFA and the fabric saddle. In one embodiment, the at least partial attachment is an at least partial permanent attachment. Permanent attachment means will be known to one of skill in the art, but may include machine stitching, circular knit stitching, knitted stitching, adhesive, binding tape and/or heat sealing through bonding or welding. Permanent attachment advantageously avoids loss or misplacing of the MMFA during laundering. Suitable means of permanent attachment will be known to those of skill in the art. In some embodiments, the attachment is by machine stitching. In other embodiments, the attachment is a seam tree adhesive or binding/tape. In some embodiments, the attachment is by machine sewing and the seam is oriented such that the bulk of the seam, including any hems, is uppermost with respect to a wearer (i.e., towards the skin of a wearer when the pant garment is in use). This orientation of the bulk of the seams/hems advantageously assists in reducing visibility of the seams/hems on the exterior of the pant garment when in use. In some embodiments, such as where the fabric saddle is composed of two or more panels of fabric sewn together, the panel seams may be used to sew in the MMFA. In other embodiments, where the fabric saddle is a single piece of circularly knitted fabric, or where the fabric saddle is composed of two or more panels joined by bonded seams, the MMFA may be attached to the saddle by any suitable means, including circular knit stitching, knitted stitching, adhesive, binding tape, and/or heat sealing through bonding or welding.

In other embodiments, the at least partial attachment is an at least partial removable attachment. Suitable means of removable attachment will be known to those of skill in the art. In some embodiments, the attachment is by hook and loop fastenings.

In one embodiment, the MMFA is at least partially attached to the fabric saddle as a single (unitary) composite fabric assembly. In other words, in such a unitary assembly, where the MMFA comprises two or more fabric layers, each layer is permanently attached to its adjacent layer. Permanent attachment of each layer may be by machine stitching, circular knit stitching, knitted stitching, adhesive, binding tape, and/or heat sealing through bonding or welding at one or more edges of each layer, or may be machine stitching or adhesive, and/or interfacing on one or more faces of each layer, or any combination of edge and/or face attachment. Examples of suitable MMFAs are included in AU2014218471B2, PCT/AU2022/050346, and PCT/AU2022/050345 the contents of each of which are incorporated herein by reference.

In one embodiment, the MMFA has opposing long edges situated between a wearer's legs proximate each leg opening of the pant garment and opposing short edges, wherein the MMFA is fixedly attached to the fabric saddle at least along the opposing long edges. In one embodiment, the MMFA adopts a generally elongate shape having opposing long edges, situated between a wearer's legs proximate each leg opening of the pant garment, and opposing short edges, and is fixedly attached to the fabric saddle along the opposing long edges only. In such embodiments, the opposing short edges are free (that is, detached from) the fabric saddle. In another embodiment, the MMFA is fixedly attached to the fabric saddle along the opposing long edges and one of the opposing short edges. In such embodiments, one of the opposing short edges is free (that is, detached from) the fabric saddle. In one embodiment, one of the opposing short edges located at the front (ventral aspect) of the MMFA is free (that is, detached from) the fabric saddle. In an alternative embodiment, one of the opposing short edges located at the back (dorsal aspect) of the MMFA is free (that is, detached from) the fabric saddle. In yet a further embodiment, the MMFA is fixedly attached to the fabric saddle along the opposing long edges and the opposing short edges. In yet another embodiment, the MMFA is fixedly attached to the fabric saddle along all its edges. In such embodiments, there may be no free edges of the MMFA free (that is, detached from) the fabric saddle. This particular embodiment requires the MMFA to be provided as a unitary composite assembly.

In some embodiments, the fabric saddle extends distalmost (underneath) the MMFA. In such embodiments, the MMFA may be at least partially attached to the fabric saddle along one or more edges of the MMFA or the MMFA may be at least partially attached to the fabric saddle though at least part of the MMFA's distalmost (undeneath) surface, or the MMFA may be at least partially attached to the fabric saddle through a combination of one or more edges of the MMFA and though at least part of the MMFA's distalmost (underneath) surface. Accordingly, in some embodiments, the MMFA is fixedly attached to the fabric saddle at one or more edges in addition to being attached on its back face. The fabric saddle extending distalmost (underneath) the MMFA advantageously offers structural support to the MMFA as well as some seam disguising capability.

In another embodiment, at least one component of the MMFA is at least partially attached to the fabric saddle. In such embodiments, as will be explained in further detail below, an MMFA “pocket” comprising a fluid barrier layer distalmost with reference to the crotch of a wearer and a moisture wicking layer proximal to the crotch of a wearer may be partially attached to the fabric saddle, with a moisture absorbent layer forming part of the MMFA being removably insertable into the pocket. It will be understood that all descriptions of MMFA attachment to the fabric saddle in the preceding paragraphs could equally apply to an MMFA pocket with a removable moisture absorbent layer, provided that at least one edge and the internal faces of the “pocket” are free to receive a removably insertable moisture absorbent layer.

The following anatomical definitions may assist understanding of the pant garments described herein. In one embodiment, a person wearing the pant garment described herein whilst standing upright has a thigh measurement, from the centre of the wearer's kneecap to an imaginary horizontal line passing in front of but in line with the wearer's crotch, of x cm. In such embodiments, a general knee area may be defined as extending from the centre of a wearer's kneecap to a position up to x/4 cm above the centre of a wearer's kneecap. In such embodiments, a general lower thigh area may be defined as extending from a position at least x/4 cm above the centre of a wearer's kneecap to a position up to x/2 cm above a wearer's knee cap. In such embodiments, a general upper thigh area may be defined as extending from a position at least x/2 cm above the centre of a wearer's kneecap to a position up to x cm above a wearer's knee cap. For completeness, a general midthigh area may be defined as extending from a position at least x/4 cm above the centre of a wearer's kneecap to a position up to 3x/4 cm above a wearer's knee cap.

The pant garments described herein generally comprise a waist portion that circumferentially extends around a torso opening of the pant garment from a front abdominal region to a lower back region. The waist portion may comprise a single fabric waistband, or it may be a comprise two or more fabric panels together comprising a waistband. The pant garments described herein generally comprise leg openings, each of which comprises a distal edge that extends circumferentially around a wearer's leg. In some embodiments, the pant garment described herein has two permanently formed leg openings. The leg openings may have any suitable length. In one embodiment, the leg openings terminate on the thigh of a wearer. In another embodiment, the leg openings terminate at the ankle of a wearer. It will be understood that there may be other embodiments where the leg openings terminate at some position intermediate between the thigh and the ankle of a wearer, or embodiments where the leg openings terminate at different positions to each other, and all such pant garments are contemplated by the disclosure herein.

In some embodiments, the pant garments described herein are leggings or tights. In such embodiments, the pant garments comprise leg openings, each of which comprises a distal edge that extends circumferentially around a wearer's leg below the wearer's knee. In one such embodiment, the distal edge of the leggings or tights on each leg independently or on both legs is positioned circumferentially around a wearer's ankle or lower leg (shin) area.

In one embodiment, at least a portion of the fabric saddle boundary is located proximate to a general knee area of the pant garment. In one embodiment, at least a portion of the fabric saddle boundary is located proximate to a general lower thigh area of the pant garment. In one embodiment, at least a portion of the fabric saddle boundary is located proximate to a general knee area and at least a portion of the fabric saddle boundary is located proximate to a general lower thigh area of the pant garment. In some embodiments, a portion of the fabric saddle boundary is located proximate to a general upper thigh area of the pant garment. In one embodiment, at least a portion of the fabric saddle boundary is located proximate to a general knee area, at least a portion of the fabric saddle boundary is located proximate to a general lower thigh area, and at least a portion of the fabric saddle boundary is located proximate to a general upper thigh area of the pant garment.

In some embodiments, a portion of the fabric saddle boundary is located proximate to a general knee area on its inner (medial) face. In some embodiments, a portion of the fabric saddle boundary is located proximate to a general lower thigh area on its front (ventral) face. In some embodiments, a portion of the fabric saddle boundary is located proximate to a general lower thigh area on its back (dorsal) face. In some embodiments, a portion of the fabric saddle boundary is located proximate to a general upper thigh area on its outer (lateral) face. In one embodiment, the fabric saddle has extremities that extend in length internally into each leg opening to a fabric saddle boundary having portions located proximate to an inner (medial) face of a general knee area, proximate to the front (ventral) and its back (dorsal) face of a general lower thigh area, and proximate to an outer (lateral) face of a general upper thigh area. In such embodiments, the fabric saddle boundary may partially “wrap” around a wearer's thigh. Such embodiments may be particularly applicable to longer pant garments, such as tights or leggings, that extend below a wearer's knee. In one embodiment, the fabric saddle has extremities that extend in length from a fabric saddle boundary at a waistband partially situated in at least a front abdominal region of the torso opening of the pant garment internally into each leg opening down an outer (lateral) face of a general upper thigh area, spanning across a front (ventral) face of a general lower thigh area to an inner (medial) face of a general knee area of the pant garment. In another embodiment, the fabric saddle has extremities that extend in length from a fabric saddle boundary at a waistband partially situated in at least a lower back or upper buttock region of the torso opening of the pant garment internally into each leg opening down an outer (lateral) face of a general upper thigh area, spanning across a back (dorsal) face of a general lower thigh area to an inner (medial) face of a general knee area of the pant garment. In such embodiments, the fabric saddle boundary may terminate along each of the ventral and dorsal faces at an edge curving from an outer (lateral) face of a general upper thigh area to an inner (medial) face of a general knee area of the pant garment. In a further embodiment, the fabric saddle has extremities that extend in length from a fabric saddle boundary at a waistband partially situated in at least a front abdominal region of the torso opening of the pant garment internally into each leg opening down an outer (lateral) face of a general upper thigh area, spanning across a front (ventral) face of a general lower thigh area to an inner (medial) face of a general knee area of the pant garment and extremities that extend in length from a fabric saddle boundary at a waistband partially situated in at least a lower back or upper buttock region of the torso opening of the pant garment internally into each leg opening down an outer (lateral) face of a general upper thigh area, spanning across a back (dorsal) face of a general lower thigh area to an inner (medial) face of a general knee area of the pant garment, terminating along each of the ventral and dorsal faces at an edge curving from an outer (lateral) face of a general upper thigh area to an inner (medial) face of a general knee area of the pant garment.

It will be understood that the particular fabric saddle boundary arrangement is not particularly limited provided that it is distally located from the crotch region. The above description represents one out of many possible arrangements of a fabric saddle boundary arrangement for the pant garments described herein. By way of non-limiting example, other suitable fabric saddle boundary arrangements could incorporate, in place of an edge curving from an outer (lateral) face of a general upper thigh area to an inner (medial) face of a general knee area of the pant garment, a wavy, zig-zag, straight line, or irregular edge that has different start and/or end points to the outer (lateral) face of a general upper thigh area and the inner (medial) face. In other embodiments, the pant garments described herein are shorts, or in particular, cycle shorts. Such shorts are also known in the art as “bike shorts”. In such embodiments, the pant garments comprise leg openings, each of which comprises a distal edge that extends circumferentially around a wearer's leg above the wearer's knee. In one such embodiment, the distal edge of the cycle shorts on each leg independently or on both legs is positioned circumferentially around a wearer's general midthigh area.

In one embodiment, at least a portion of the fabric saddle boundary extends along a general thigh area of the pant garment. In one embodiment, at least a portion of the fabric saddle boundary is located proximate to a general upper thigh area of the pant garment. In some embodiments, a portion of the fabric saddle boundary is located proximate to a general midthigh area of the pant garment. In one embodiment, at least a portion of the fabric saddle boundary extends from a position proximate to a general upper thigh area to a position proximate to a general midthigh area of the pant garment.

In one embodiment, the fabric saddle has extremities that extend in length internally into each leg opening to a fabric saddle boundary located proximate to a general upper thigh area on its front (ventral) face. In some embodiments, the fabric saddle has extremities that extend in length internally into each leg opening to a fabric saddle boundary located proximate to a general upper thigh area on its back (dorsal) face. In one embodiment, the fabric saddle has extremities that extend in length from a fabric saddle boundary at a waistband partially situated in at least a front abdominal region of the torso opening of the pant garment internally into each leg opening along a front (ventral) face of a general upper thigh area to a front (ventral) face of a general midthigh area of the pant garment. In another embodiment, the fabric saddle has extremities that extend in length from a fabric saddle boundary at a waistband partially situated in at least a lower back or upper buttock region of the torso opening of the pant garment internally into each leg opening along a back (dorsal) face of a general upper thigh area to a back (dorsal) face of a general midthigh area of the pant garment. In such embodiments, the fabric saddle boundary may terminate at a distal edge of the pant garment positioned circumferentially around a wearer's leg above the wearer's knee. In a further embodiment, the fabric saddle has extremities that extend in length from a fabric saddle boundary at a waistband partially situated in at least a front abdominal region of the torso opening of the pant garment internally into each leg opening along a front (ventral) face of a general upper thigh area to a front (ventral) face of a general midthigh area of the pant garment and extremities that extend in length from a fabric saddle boundary at a waistband partially situated in at least a lower back or upper buttock region of the torso opening of the pant garment internally into each leg opening along a back (dorsal) face of a general upper thigh area to a back (dorsal) face of a general midthigh area of the pant garment, terminating at a distal edge of the pant garment positioned circumferentially around a wearer's leg above the wearer's knee. In such embodiments, the fabric saddle boundary may wrap the inside (medial) face of each of a wearer's thighs. Such embodiments may be particularly applicable to shorter pant garments, such as cycle shorts, that end above a wearer's knee.

The precise shape of the fabric saddle is not particularly limited, provided that fabric saddle boundary is distally located from the crotch region. The skilled person will appreciate that there are many design choices that may be made in the positioning of the fabric saddle boundary, such as to accentuate muscle lines, hip or thigh curvature, etc. or to embellish the pant garment with distinctive or attractive lines and/or patterns. In some embodiments, the fabric saddle extends down into leg openings in the pant garment such that the fabric saddle hugs a wearer's inner thigh in use. In other embodiments, the fabric saddle extends down into leg openings in the pant garment such that the fabric saddle wraps around a wearer's thigh in use. Suitable locations for the fabric saddle boundary distally located from the crotch region will be clear to a person of skill in the art and may vary with the type of garment (shorts vs. leggings).

In one embodiment, the fabric saddle has extremities that extend in length from a fabric saddle boundary at a waistband partially situated in at least a front abdominal region of the torso opening of the pant garment on a wearer's ventral face, through the crotch region between a wearer's legs and either extending under the MMFA or in parts attached to a leading and a trailing edge of the MMFA, through to a fabric saddle boundary at a waistband partially situated in at least a lower back or upper buttock region of the torso opening of the pant garment on a wearer's dorsal face. In such embodiments, the fabric saddle boundary is fixedly attached in the waistband portion of the garment. In some embodiments, the fabric saddle boundary is fixedly attached only in the waistband portion of the garment and the part of the fabric saddle boundary that extends between the waistband at the front abdominal region and the lower back or upper buttock region is free (i.e., unattached) to the interior of the pant garment. In this embodiment, the fabric saddle adopts more of a fabric hammock shape.

In one embodiment, the fabric saddle comprises two or more fabric pieces connected together. Such a construction is advantageously mechanically convenient to produce as it can utilise one or more joins between the fabric pieces to create a curving saddle shape. In some embodiments, the joins are seams, such as machine stitched seams. In other embodiments, the joins are seam free, such as connected by adhesive tape or fused together. In other embodiments, the fabric saddle may instead be a single piece of fabric, formed by a technique such as circular knitting.

In some embodiments, the fabric saddle is fixedly attached to the interior surface of the pant garment solely along a part or full length of the fabric saddle boundary and remains unattached to the interior surface of the pant garment along all other surfaces of the fabric saddle. In other words, the fabric saddle is “floating” or completely free from the pant garment at all points except at the fabric saddle boundary. This allows physical separation of the fabric saddle (and hence partially attached MMFA) from the interior surface of the pant garment at the crotch region in particular. The fabric saddle boundary will generally have a portion located at a front abdominal aspect of the pant garment up and a portion at a back upper buttock/lower back aspect of the pant garment. These portions of the fabric saddle boundary will generally be connected by a portion of the fabric saddle extending in a general hammock shape through the crotch region between a wearer's legs. The fabric saddle boundary will also generally have a portion located in each leg opening of the pant garment, in some embodiments running from the torso opening (waist) of the pant garment along a front (ventral) surface of a wearer's thighs in use, in other embodiments running along a back (dorsal) surface of a wearer's thighs in use, and in yet other embodiments running along a side (lateral) surface of a wearer's thighs in use, or in yet further embodiments, running along a combination of two or more of the aforementioned surfaces (either on each leg opening or on the leg openings collectively). The floating fabric saddle advantageously allows physical separation of the MMFA from the interior of the garment, which in turn may improve access to drying air and therefore improve the air-drying times of these products. Faster drying products are more favoured by the consumer and also are associated with reduced risk of mould growth and malodors.

It will also be understood that the fabric saddle described herein may be fixedly attached along all or part of the fabric saddle boundary to the interior surface of the pant garment. By way of non-limiting example, the fabric saddle boundary may be fixedly attached to an interior surface of the pant garment at all points along the boundary from the waistband and down the leg openings but be unattached from the interior surface of the pant garment at the cuffs, such as cuffs at the thigh, knee, calf or ankle. In other embodiments, the fabric saddle boundary is fixedly attached to an interior surface of the pant garment at all points along the boundary from the waistband and down the leg openings and including at the cuffs. The fabric saddle boundary at the cuffs, whether attached or unattached to an interior surface of the pant garment, may have any suitable shape. In some embodiments, the fabric saddle boundary at the cuffs has a straight substantially horizontal edge, or may have a straight slanted edge, or a curved or wavy edge that sits substantially horizontally or that sits at any suitable angle, or an irregular edge having straight and curved elements. In yet other embodiments, the fabric saddle boundary may be fixedly attached to an interior surface of the pant garment at regular or irregular intervals from the waistband down the leg openings and optionally also at the cuffs, such as being fixedly attached at regular or irregular intervals of any suitable dimension, by way of non-limiting example only, at regular or irregular intervals of between 0.1 and 30 cm, such as 2 cm, 4 cm, 6 cm, 8 cm, 10 cm, up to 20 cm, up to 30 cm, with regular or irregular intervals of non-attachment in between. In this way, it is envisaged that pant garments with a fabric saddle boundary attached to an interior surface of a pant garment with one or more gaps or points of detachment between portions of the fabric saddle boundary attached to the interior of the pant garment are within the scope of the present invention.

The means by which the fabric saddle is fixedly attached to the interior surface of the pant garment is not particularly limited. The term “fixedly attached” will be understood to mean permanent, secure attachment of the fabric saddle to the interior surface of the pant garment. Suitable means of fixed attachment will be known to those of skill in the art, but may include machine stitching, circular knit stitching, knitted stitching, adhesive, binding tape, and/or heat sealing through bonding or welding. In some embodiments, the fixed attachment is by machine stitching. In other embodiments, the fixed attachment utilises a seam free adhesive or binding/tape or bonding or welding technique. In some embodiments, the fixed attachment is by machine sewing and the seam is oriented such that the bulk of the seam, including any hems, is innermost with respect to a wearer (i.e., faces towards the inside of the pant garment and towards the skin of a wearer when the pant garment is in use). In some embodiments, the fabric saddle may be fixedly attached to an interior surface of the pant garment at the torso opening of the pant garment at a waistband by circular knit stitching, cover stitching, knitted stitching and/or heat sealing through bonding or welding. In such embodiments, a double layer waistband construction may “sandwich” the fabric saddle boundary between waistband layers and allow for a neat finish by stitching (including knit stitching, cover stitching or circular knit stitching) or heat bonding.

In one embodiment, the fabric saddle comprises one or more layers of a fabric. In one embodiment, the fabric saddle comprises one or more layers of fabric in which at least one layer is a fabric mesh. The fabric of the fabric saddle may comprise one or more layers of a natural fabric, a synthetic fabric, a semi synthetic fabric or a combination thereof. In one embodiment, the fabric of the fabric saddle comprises one or more layers of a synthetic fabric. In one embodiment, the fabric of the fabric saddle comprises a synthetic nylon/spandex blend. In various embodiments, the fabric saddle may comprise one layer of fabric, or may comprise one layer of a synthetic fabric, or may comprise one layer of a synthetic nylon/spandex blend. In one embodiment, the fabric of the fabric saddle may be treated to augment its properties, for example, treated with a cooling treatment, an anti-odour treatment, or a wicking treatment. In one embodiment, the fabric of the fabric saddle may be treated with a wicking treatment that increases the moisture absorbency of the fabric and assists with evaporation from the fabric.

Commercially available wicking treatments are known in the art. Such wicking treatments may include treatments sold by Midori-Bio Inc. as miDori® bioWick or bioDry. In another embodiment, the fabric saddle comprises a cellulosic material. In some embodiments, the fabric saddle comprises a cellulosic material selected from cotton, a cotton blend, bamboo, wool, micromodal, or lyocell (brand name Tencel®), or a blend comprising any of these fabrics. In another embodiment, the fabric saddle comprises a biodegradable or compostable cotton, a cotton blend, bamboo; wool; micromodal, or lyocell (brand name Tencel®) or blend of any of these fabrics. In a further embodiment, the fabric saddle comprises a biobased cotton, a cotton blend, bamboo; wool; micromodal, or lyocell (brand name Tencel®) or blend of any of these fabrics.

As used herein, the term “fabric mesh” refers to a fabric whose structure comprises open spaces or holes. In some embodiments, a “fabric mesh” refers to a fabric which has a woven or knitted arrangement of yarn or fibres. In some preferred embodiments, the fabric mesh comprises a multiplicity of regular open spaces or holes between the yarns. Mesh fabrics can be woven or knitted or formed using related techniques such as used in crochet, lace, or knotting.

The size of the “holes” will be defined by the tension used during fabric formation. In some embodiments, the fabric mesh comprises between 6 and 16 holes per inch. Examples of mesh include knitted or woven fabrics or more complex mesh structures such as power mesh or cool mesh, but other mesh fabrics suitable for use as a fabric saddle as described herein will be known to those of skill in the art. In one embodiment, the mesh is a lightweight fabric of between 60 and 160 gsm and having four-way stretch. In one embodiment, the mesh is a lightweight fabric resembling stretchy netting and having four-way stretch. In one embodiment, the fabric mesh may be laser cut at its edges. In one embodiment, the fabric saddle comprises a layer of fabric mesh. In another embodiment, the fabric saddle consists of a layer of fabric mesh. In one embodiment, the fabric mesh comprises a nylon blend or composite fabric. In one embodiment, the fabric mesh comprises a blend of nylon and spandex (elastane). In one embodiment, the fabric mesh comprises a blend of between 90-95% by weight nylon and 5-10% spandex, or a blend of 93% nylon and 7% spandex. In one embodiment, the nylon is recycled nylon. In other embodiments, the fabric mesh may comprise a biodegradable polymer, or a compostable polymer, or a biobased polymer, or a blend thereof. In some embodiments, the fabric mesh may comprise a synthetic fabric. In other embodiments, the fabric mesh may comprise a natural fabric. In other embodiments, the fabric mesh may comprise a semi-synthetic fabric. In one embodiment, the fabric mesh may be treated to augment its properties, for example, treated with a cooling treatment, an anti-odour treatment, or a wicking treatment. In some embodiments, the fabric mesh is treated with a wicking treatment, such as sold by Midori-Bio Inc. as miDori® bioWick or bioDry. Fabric mesh products are commercially available, including from HTT as the product Power Mesh. A fabric mesh having any suitable gsm may be used. In some embodiments, fabric mesh of between 60 and 160 gsm may be used, or a fabric mesh of between 80 gsm and 120 gsm may be used, or a fabric mesh of between 90 gsm and 110 gsm may be used, or a fabric mesh of between 100 and 105 gsm may be used.

Any type of MMFA known to the skilled person can be used in the present invention. However, a preferred MMFA as described herein may comprise at least a fluid wicking layer, and a fluid barrier layer. Additional functional layers can be included as needed. For example, a preferred MMFA may comprise a fluid wicking layer, a fluid absorbing fabric core, and a fluid barrier layer. Mostly these components are fabric components however, the fluid barrier layer may be a fluid barrier web or laminate provided on a distinct fabric component or provided on the underside of the fluid wicking layer or the core when present. In one embodiment, the MMFA as described herein comprises three parts: a fluid absorbing fabric core, a fluid barrier layer, and a fluid wicking layer. In other embodiments, the MMFA as described herein comprises two parts: a fluid absorbing fabric core and a fluid barrier layer or a fluid wicking layer. In one embodiment, the MMFA is structured to have the fluid wicking layer uppermost (closest to a wearer's skin in use), a fluid barrier layer lowermost (furthest from a wearer's skin in use), and a fluid absorbing fabric core sandwiched between the fluid barrier layer and the fluid wicking layer. Each of these parts will be described below.

The MMFA may have a total thickness of between 1 and 25 mm, or between 1 and 15 mm, or between 1 and 10 mm, or between 1 and 9 mm, or between 1 and 7 mm, or between 1 and 5 mm, or between 1 and 2 mm. In one embodiment, the MMFA comprises up to 5 individual fabric layers and has an overall thickness of between 1 and 10 mm. In some embodiments, preferred MMFAs have a total thickness of between 1 and 5 mm, more preferably 1 and 2 mm for additional discreetness. In one embodiment, the MMFA has a thickness of <5 mm, or less than 4 mm, or less than 3 mm, or less than 2 mm, or less than 1 mm.

In one embodiment, the MMFA comprises a fluid absorbing fabric core for trapping bodily fluid secretions. The fluid absorbing fabric core may include one or more layers of a fluid absorbing fabric or material such as a cotton or cotton blend padding. In one embodiment, the fluid absorbing fabric core comprises at least 1, at least 2, at least 3, at least 4 or at least 5 layers of fluid absorbing fabric, or comprises 1, 2, 3, 4 or 5 layers of fluid absorbing fabric, or comprises up to 6, up to 5, up to 4, up to 3, or up to 2 layers of fluid absorbing fabric. In one embodiment, the fluid absorbing fabric core comprises 3 layers of fluid absorbing fabric. Each layer of fabric in the fabric absorbent core may be the same. In some embodiments, the fabric absorbent core may comprise two or more different fabric layers, that is, may comprise a combination of different fabric layers. The moisture management pant garment described herein may be capable of managing low, moderate and/or moderate-high moisture levels by utilising a number of layers of absorbent fluid absorbent fabric, preferably microfibre terry fabric in a fluid absorbing fabric core provided in the crotch region of the garment.

Suitable fluid absorbing fabrics for use in the fluid absorbing core will be known to those of skill in the art. In one embodiment, the fluid absorbing fabric is a quick drying absorbent fabric. In some embodiments, fluid absorbing fabric comprises a synthetic fabric (derived from manmade fibres), a semi-synthetic fabric (being a synthetically modified derivative of a material of natural origin and including cellulosic fibres such as Modal) or a natural fabric (derived from fibres of 100% natural origin). In some embodiments, the fluid absorbing fabric is a biodegradable, biobased, or compostable version of any of the afore- and below-mentioned fabrics. Accordingly, in one embodiment, the fluid absorbing fabric core comprises a partly or fully bio-based absorbent quick drying fabric, a biodegradable absorbent quick drying fabric, or a compostable absorbent, quick drying fabric. As used herein, the term “biodegradable” is used in the sense of being at least 80% compostable but more preferably at least 95% to 100% compostable in active soil in 5 years or less, preferably in 4 years or less, preferably in 3 years or less, more preferably in 2 years or less, and most preferably in 1 year or less. Active soil comprises typical soil microbes including bacteria and fungi. As used herein, the term “biobased” in relation to fabric refers to the source of raw materials for the fabric being plant-based, such as from corn or hemp rather than fossil-fuel based. As used herein, the term “compostable” refers to materials that readily break down in a composting system through the action of microbial activity in a defined time period to form compost or humus comprising natural materials only that are safe for the environment. The defined time period may be in 5 years or less, in 4 years or less, in 3 years or less, more preferably in 2 years or less, and most preferably in 1 year or less.

In some embodiments, the fluid absorbing fabric is a quick drying microfibre fabric, or is a polyester microfibre fabric, or is an absorbent, quick drying fabric manufactured from a cellulosic material. In some embodiments, the cellulosic material is hemp, cotton or bamboo. In some embodiments, the fluid absorbing fabric comprises a microfibre towelling comprising greater than 50% polyester and less than about 50% polyamide, or a microfibre towelling that is a bamboo terry, as this material is biodegradable/compostable in active soil. In one embodiment, the the fluid absorbing fabric is a microfibre fabric comprising a polyester/polyamide blend, such as 90% polyester/10% polyamide, or 85% polyester/15% polyamide, or 80% polyester/20% polyamide, or 70% polyester/30% polyamide. Suitably, the fluid absorbing fabric may comprise a microfibre towelling comprising about 80% polyester and about 20% polyamide. In some embodiments, the polyester is a recycled polyester. In some embodiments, the polyamide is a recycled polyamide. In some embodiments, the fluid trapping core component comprises a microfibre fabric comprising recycled polyester/recycled polyamide blend, such as 90% recycled polyester/10% recycled polyamide, or 85% recycled polyester/15% recycled polyamide, or 80% recycled polyester/20% recycled polyamide, or 70% recycled polyester/30% recycled polyamide. In some embodiments, the fluid absorbing fabric is a polyamide microfibre fabric or polyamide blend microfibre fabric such as a polyamide/elastane blend fabric, or a polyester blend microfibre fabric such as a polyester/polyamide blend microfibre fabric; or an absorbent, quick drying fabric manufactured from a cellulosic material, preferably cotton, Tencel, hemp or bamboo, optionally wherein the cellulosic material is treated to improve absorbency; or a natural fabric such as wool, especially Merino wool, most preferably a polyester/polyamide blend microfibre fabric.

In some embodiments, the fluid absorbing fabric comprises a cellulose-based fabric. In some embodiments, the fluid absorbing fabric is treated to augment its properties, for example, treated with a cooling treatment, an anti-odour treatment, and/or a wicking treatment. In one embodiment, the fluid absorbing fabric comprises a cellulose-based fabric with a quick dry or wicking treatment that increases the moisture absorbency of the fabric. Commercially available wicking treatments are known in the art. Such wicking treatments may include treatments sold by Midori-Bio Inc. as miDori® bioWick or bioDry. In some embodiments, the fluid absorbing fabric includes an odour elimination treatment. In another embodiment, the fluid absorbing fabric comprises a cellulose-based fabric with an anti-odour treatment. Suitable anti-odour or odour elimination treatments may include Sciessent Lava XL and Agion. In some embodiments, suitable anti-odour or odour elimination treatments are bio-based.

In one embodiment, the fluid absorbing fabric core comprises up to 5 layers, up to 4 layers, or up to 3 layers of an absorbent, quick drying fabric. A preferred fabric core may have 1, 2 or 3 layers of fluid absorbing fabric. In one embodiment, the fluid absorbing fabric core comprises up to 3 layers of an absorbent, quick drying microfibre fabric including a bamboo terry, a polyester, a recycled polyester or biodegradable polyester or a biobased polyester, or a polyester composite microfibre fabric, including a polyester/polyamide blend. In another embodiment, the fluid absorbing fabric core comprises 3 layers of an absorbent, quick drying microfibre fabric including a bamboo terry, a polyester, a recycled polyester or biodegradable polyester or a biobased polyester, or a polyester composite microfibre fabric, including a polyester/polyamide blend. Each of the layers need not have identical dimensions. Accordingly, in some embodiments, a second and/or third layer. when present, may have a smaller area (i.e., smaller length and/or width) than a first (or bottom/distal) layer of the fluid absorbing fabric core.

Each layer of fluid absorbent fabric may have a weight or density in a range of 50 gsm to 500 gsm, or between 150 gsm and 350 gsm, or between 220 gsm and 260 gsm, or a weight or density of 240 gsm.

The fluid absorbing fabric core may have a thickness of between 1 and 25 mm, 1 and 15 mm, 1 and 10 mm, 1 and 9 mm, 1 and 7 mm, 1 and 5 mm, or 1 and 2 mm. In one embodiment, the fluid absorbing fabric core comprises up to 3 layers and has a thickness of between 1 and 10 mm, such as a thickness of between 1 and 5 mm.

The fluid absorbing fabric core may have a fluid absorbency of up to 250 mL, or of up to 200 mL, or of up to 150 mL, or of up to 100 mL, or of up to 90 mL, or of up to 80 mL, or of up to 70 mL, or of up to 60 mL, or of up to 50 mL, or of up to 40 mL, or of up to 30 mL, or of up to 20 mL, or of up to 10 mL. In one embodiment, the fluid absorbing fabric core has a fluid absorbency of between 0.1 and 50 mL. In one embodiment, the fluid absorbing fabric core has a fluid absorbency of between 0.1 and 250 mL. In one embodiment, the fluid absorbing fabric core has a fluid absorbency of between 10 and 250 mL.

As described above, the MMFA may be a single (unitary) composite assembly or may be an MMFA “pocket” comprising a fluid barrier layer distalmost with reference to the crotch of a wearer and a fluid wicking layer proximal to the crotch of a wearer, with the fluid absorbing fabric core being removably insertable into the pocket. This may allow the fluid absorbing fabric core component to be replaced and laundered or replaced separately. However, in other embodiments, the fluid absorbing fabric core component is a permanent part of the MMFA. This may be by stitching, adhesive, or other means of attachment of the fluid absorbing fabric core inside the pocket, either to one or more faces or one or more edges of the pocket, or may be by sealing the pocket at its edges by stitching, adhesive, or other means with the fluid absorbing fabric core already inside it, such that the fluid absorbing fabric core is unable to be removed from the pocket. This avoids the possibility of losing the fluid absorbing fabric core during laundering.

At least a portion of the MMFA described herein may comprise a fluid wicking fabric. In one embodiment, the MMFA comprises a fluid wicking fabric for directly contacting a wearer's skin in use, wherein the fluid wicking fabric is positioned adjacent to a fluid absorbing fabric core. The fluid wicking fabric may be provided on the skin facing side of the MMFA, where direct contact with the skin allows any moisture to be quickly wicked away. Indeed, the fluid wicking fabric may assist in wicking bodily fluids, including sweat, urine, blood and/or discharge, quickly away from a wearer's skin into the fluid absorbing fabric core of the MMFA in the pant garment described herein.

The fluid wicking fabric may be selected from cotton; a cotton blend such as a cotton elastane jersey, bamboo fleece; wool such as Merino wool; wicking polyester, wicking polypropylene, wicking nylon, wicking Modal, wicking micromodal, wicking lyocell (brand name Tencel®), or wicking bamboo; a brushed fabric having a brushed or otherwise textured surface having fibres displaced from the fabric knit, such as a brushed wool, a brushed Merino wool, a brushed wicking polyester, a brushed wicking polypropylene, a brushed wicking nylon, a brushed wicking micromodal, a brushed wicking bamboo, a brushed cotton, or a composite or blend thereof. In one embodiment, the fluid wicking fabric is a brushed fabric. In one embodiment, the fluid wicking fabric is a brushed wool, brushed polyester, brushed polypropylene, brushed nylon, brushed micromodal, brushed bamboo, brushed cotton, or a composite or blend thereof. In one embodiment, the fluid wicking fabric is a brushed wool. In one embodiment, the fluid wicking fabric of the MMFA is brushed Merino wool. In another embodiment, the moisture wicking fabric is a bamboo fabric or bamboo fleece. In a further embodiment, the moisture wicking fabric is a biodegradable bamboo fabric or biodegradable bamboo fleece. In one embodiment, a cotton/elastane jersey may be used, where the cotton/elastane jersey knit comprises a blend of cotton and elastane, such as 80% cotton/20% elastane, 85% cotton/15% elastane, or 90% cotton/10% elastane. In some embodiments, the elastane is biobased elastane, being wholly or in part derived from a natural source such as a plant source, e.g., corn.

Suitably, the fluid wicking fabric has a weight or density in a range of about 100 gsm to about 300 gsm, between about 150 gsm and about 250 gsm, between about 180 gsm and about 200 gsm, or a weight or density of about 190 gsm.

In some embodiments, the fluid wicking fabric is treated to augment its properties, for example, treated with a cooling treatment, an anti-odour treatment, an antimicrobial treatment and/or a wicking treatment. In some embodiments, the fluid wicking fabric includes an antimicrobial material or treatment. Antimicrobial materials or treatments are known in the art, and include silver ions. In some embodiments, the fluid wicking fabric includes an odour elimination treatment. Suitable treatments may include Sciessent Lava XL and Agion or bio-based equivalents thereof. In other embodiments, the fluid wicking fabric itself comprises fibres having antimicrobial properties, such as wool, including soft wool such as Merino wool. It will be understood that an antimicrobial material is one that has antibacterial, antiviral, and/or antifungal properties. The antimicrobial property minimises, and more preferably eliminates, odour arising from the bodily fluid.

At least a portion of the MMFA described herein may comprise a fluid barrier fabric or fluid barrier polymer web. In another embodiment, the MMFA comprises a fluid barrier fabric which is preferably breathable to air and moisture vapour but not liquid, to prevent fluid leaking outside the pant during use, wherein the fluid barrier fabric is positioned adjacent to a fluid absorbing fabric core. The fluid barrier fabric may be provided on the outer facing side of the MMFA, that is, located distal to a wearer's skin in use, where it can prevent egress of fluid from the fluid absorbing fabric core (optionally through the fabric saddle layer) to the outer wear garment shell/outer layer. In some embodiments, the fluid barrier fabric is fixedly attached to the MMFA and positioned between the fluid absorbing layer and the fabric saddle. In one embodiment, the fluid barrier fabric is provided between the fabric saddle (adjacent to the outer wear garment shell/outer layer fabric) and the fluid absorbing fabric core. In one embodiment, the fluid barrier fabric is provided between the outer wear garment shell/outer layer fabric and the fluid absorbing fabric core.

The fluid barrier fabric may be a fluid proof fabric, a fluid resistant fabric or a fluid repellent fabric or may be a fabric treated to be fluid proof, fluid resistant or fluid repellent. A fluid proof fabric, a fluid resistant fabric or a fluid repellent fabric is advantageously capable of preventing movement of bodily fluids from the fluid absorbing fabric core to outside the pant, thereby avoiding leakage of captured bodily fluid.

In one embodiment, the fluid barrier component is a fabric which comprises a waterproof or water-resistant laminate film or a chemical coating, rendering the fabric fluid proof, fluid resistant and/or fluid repellent. In another embodiment, the fluid barrier component comprises a fabric which is itself fluid proof, fluid resistant or fluid repellent. In one embodiment the fabric is a fabric treated with one or more agents including hydrophobic polymers, such as silicon derived polymers like silicone or siloxane polymers, polyethylene based hydrophobic polymers, hydrophobic non-fluorinated acrylic polymers, hydrophobic non-fluorinated urethanes, or bio-based or bio-derived hydrophobic polymers or compounds, (ideally not comprising fluorinated compounds such as PFOAs), which may assist with waterproofing, water resistance or water repellence. Suitable fluid barrier fabrics may be treated with water proofing or water repelling agents such as those available from the companies Huntsman or Rudolf. A preferred agent used to treat fabric to impart waterproof or water resistant/repellency qualities include Aquapel™ by Nanotex which is a water repellent polymer based composition. Suitable fluid barrier fabrics having inherent waterproofing or water resistance or water repellency are available from Ding Zing. It will be understood that the term “water” is used herein in the sense that it encompasses bodily fluids, particularly, sweat, urine, discharge, and/or blood.

In other embodiments, the fluid barrier fabric comprises a polyester/polyurethane laminate fabric. In one embodiment, the polyester/polyurethane laminate is the form of a cotton or polyester knit fabric bonded to a polyurethane breathable and waterproofing film, generally having a thickness of 1 mm or less. The fluid barrier fabric may be recycled and/or biodegradable. In one embodiment, the fluid barrier fabric is recycled and/or biodegradable polyester and biodegradable polyurethane which may be laminated directly onto the fluid absorbing fabric core. In other embodiments, the fluid barrier component comprises a biodegradable and/or compostable polymer film alone, such as a thermoplastic polyurethane (TPU) film, e.g., available from Ding Zing. Such films can be used wherever water barrier function is required, for example, it can be laminated directly to the underside of the fluid absorbing fabric core.

In a further embodiment, the fluid barrier fabric comprises a polyester fabric. In one embodiment, the fluid barrier fabric comprises recycled polyester. In one embodiment, the fluid barrier fabric comprises a biobased polyester fabric.

Suitably, the fluid barrier fabric has a weight or density in a range of about 100 gsm to about 300 gsm, between about 120 gsm and about 220 gsm, between about 150 gsm and about 200 gsm, or a weight or density of about 175 gsm.

In one embodiment, the pant garment herein is in the form of tights, leggings or shorts, including cycle shorts. In one embodiment, the pant garment herein is in the form of tights or leggings. In another embodiment, the pant garment herein is in the form of shorts or cycle shorts. Other form fitting garments may also utilise the fabric saddle described herein.

In one embodiment, the pant garment herein has a fabric shell or outer layer. Although suitable fabric shell or outer layer fabrics for outer wear will be known to those of skill in the art, the shell or outer layer fabric may be polyester, nylon, cotton, hemp, Tencel, bamboo viscose, or bamboo Lyocell; or a biobased, or biodegradable or compostable, or recycled version of any one of these; or a blend comprising any of the afore-listed fabrics, including elasticised blends. In one embodiment, a material suitable for forming elasticized blends is elastane. In some embodiments, the elastane is biobased elastane, being wholly or in part derived from a natural source such as a plant source, e.g., corn. In one embodiment, a material suitable for forming elasticized blends is spandex or lycra. In one embodiment, the shell or outer layer fabric is a nylon/spandex blend, or a 70-90% nylon/30-10% spandex blend, or a 80% nylon/20% spandex blend, or recycled nylon/spandex blend, or a 70-90% recycled nylon/30-10% spandex blend, or a 80% recycled nylon/20% spandex blend. In some embodiments, the shell or outer fabric is treated to augment its properties, for example, treated with a cooling treatment, an anti-odour treatment, an antimicrobial treatment and/or a wicking treatment. In some embodiments, the shell or outer layer fabric may be treated with a wicking treatment that increases the moisture absorbency of the fabric and assists with moisture evaporation from the fabric. Commercially available wicking treatments are known in the art. Such wicking treatments may include treatments sold by Midori-Bio Inc. as miDori® bioWick or bioDry. The shell fabric may be provided with any desired pattern or colour scheme. The shell or outer fabric may be treated to increase stain resistance. Commercially available treatments to increase stain resistance include Scotchguard™ or Teflon™.

In some embodiments, the shell or outer layer of the pant garment comprises a crotch shaping panel for ease of fit of the pant garment. In particular, the crotch shaping panel may assist in fitting and/or ensuring the correct sit of the MMFA in the crotch region of the pant garment, and/or it may avoid the tendency of the garment to gather in the female contours and reveal the imprint or impression of the labia majora. As described herein, the crotch shaping panel, if present, is positioned in the crotch region and generally concealed between a wearer's legs. In some embodiments, the crotch shaping panel in the shell of the pant garment is triangular or approximately triangular in shape, although other suitable shapes will be evident to those of skill in the art. In one embodiment, the crotch shaping panel may be generally triangular in shape, especially having a generally isosceles triangle shape, having an apex of the triangle pointed towards a wearer's dorsal face or back/bottom, and a triangle base centred and directed towards a wearer's ventral face or front/abdomen. The crotch panel may have any suitable dimensions, but will generally have dimensions, with reference to an isosceles triangle shape, wherein a triangle base is from 2-6 cm in length and a triangle height is from 4-10 cm. Accordingly, the crotch shaping panel is generally a small panel relative to the size of the crotch region. It should be noted that the crotch shaping panel is not itself a MMFA as described herein, nor does it have the same function as an MMFA as described herein. Typically, the crotch shaping panel is a single layer of fabric. It will also be understood that any external visibility of the crotch shaping panel is in no way connected to the external visibility of the MMFA. Although the crotch shaping panel as described in this paragraph may, in some embodiments, be a separate panel stitched into the crotch region of the shell or outer layer, it will generally be positioned in the crotch region between a wearer's legs where it is not visible when the pant garment is being worn when viewed from both the front and back a wearer standing up. Further, the crotch shaping panel has a different shape and size to the MMFA disposed inside the pant garment, and is therefore not an external visible indicator of an MMFA being present in the pant garment. It should also be noted that in some embodiments, the crotch shaping panel is not attached to the pant garment in a way that results in significant visible seams in the crotch area as would result from inclusion of a MMFA without the saddle as described herein. Nonetheless, for the avoidance of doubt, in some embodiments, the externally visible seams in the crotch region of the pant garment described herein exclude seams of a crotch shaping panel in an outer layer or shell of the pant garment, if one is present. In some embodiments, the crotch shaping panel may be integrally formed into the shell or outer layer of a pant garment using, e.g., circular knitting techniques and will therefore have no seams or visible seams.

In one embodiment, the pant garment herein comprises additional features such as a pocket. In one embodiment, the pocket is a suitably dimensioned to accommodate a mobile phone. Other suitable alterations or ornamentations will be known to persons of skill in the art.

The pant garment described herein is hand and/or machine washable (“launderable”) and is suitable for repeat wash-and-wear. In some embodiments, the pant garments may be launderable through at least 5, or at least 10, or at least 20, at least 50, or at least 100 washing cycles and yet still retain their moisture management functionality.

### Preferred Embodiments/Examples

The present invention is described with reference to the following examples. It is to be understood that the examples are illustrative of and not limiting to the invention described herein.

FIG. 1 illustrates a non-limiting example of a fabric saddle of the invention provided inside a pant garment and which takes the form of a fabric manufactured to have, or provided in, a generally flared hyperboloid paraboloid shape. Front and back curved portions of the paraboloid shaped fabric saddle 12 projecting above the central saddle point 11 flatten or have a steep gradient to accommodate or fit or contact against the abdominal and lower back/upper buttock areas of a wearer (depending on orientation of the saddle). Side curved portion 10 (and corresponding side curved portion in rear not shown) of the paraboloid projects below the central saddle point and in this example ultimately extend down along each leg of a wearer (not shown), optionally wrapping partially or fully around each leg by means of an asymmetric extension of the paraboloid boundary (not shown). During use a wearer's crotch region is designed to be accommodated in or near central saddle point 11, with one leg of a wearer extending down in the direction of the paraboloid side projection 10 below the central saddle point and second leg extending down in the direction of the paraboloid side projection below the central saddle point to the rear (not shown).

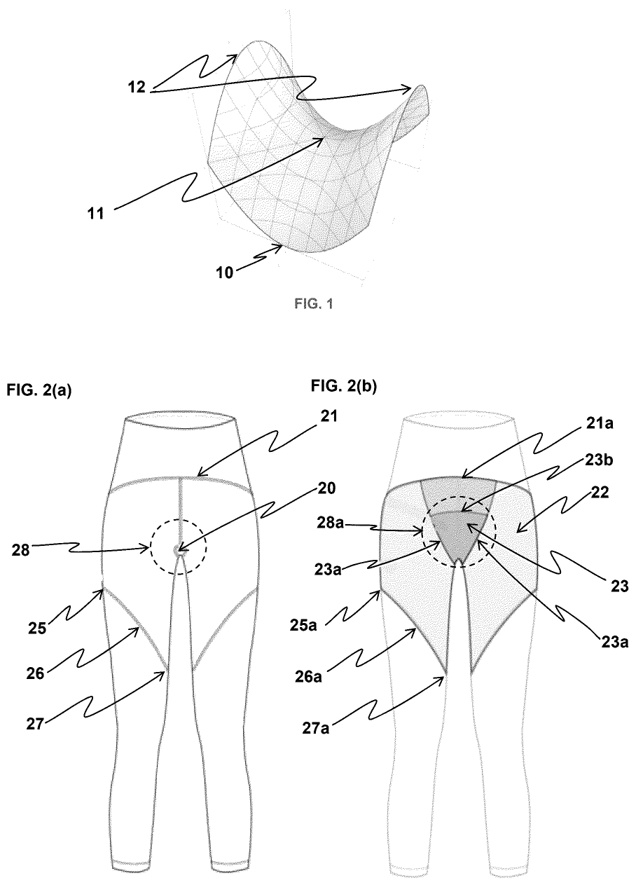

FIG. 2(a) illustrates a front external view of one preferred embodiment of a pant garment as described herein in the form of exercise leggings or exercise tights and comprising an MMFA in the crotch associated with fabric saddle. In FIG. 2(a) there is an externally visible seam or stitching 21 away from the crotch and closer to a waistband situated in a front abdominal region of the torso opening of the pant garment. There is also externally visible stitching on the outer (lateral) face of the general upper thigh area 25, spanning across the front (ventral) face of the general lower thigh area 26 to the inner (medial) face of the general knee area 27 of the pant garment. A small crotch shaping panel 20—in some embodiments triangular or approximately triangular in shape—formed from the shell/outer fabric and provided for ease of fit of the pant garment is also shown. The MMFA or any stitching or seams associated therewith is not at all visible in the crotch region as highlighted by circle 28. FIG. 2(b) illustrates a front view of the same pant garment as FIG. 2(a) but when viewed inside out. In FIG. 2(b), the fabric saddle 22 has extremities that extend in length from a fabric saddle boundary close to the waistband situated in a front abdominal region of the torso opening of the pant garment 21a internally into each leg opening down the outer (lateral) face of the general upper thigh area 25a. The extremities span across the front (ventral) face of the general lower thigh area 26a to the inner (medial) face of the general knee area 27a of the pant garment. The MMFA 23 is located in the crotch region of the pant garment of FIG. 2(b). In one embodiment, the MMFA is partially attached to the fabric saddle along its generally long edges 23a and free (unattached to the fabric saddle) along its short edge 23b. In this inner view, the MMFA is visible in the crotch region as highlighted by circle 28a.

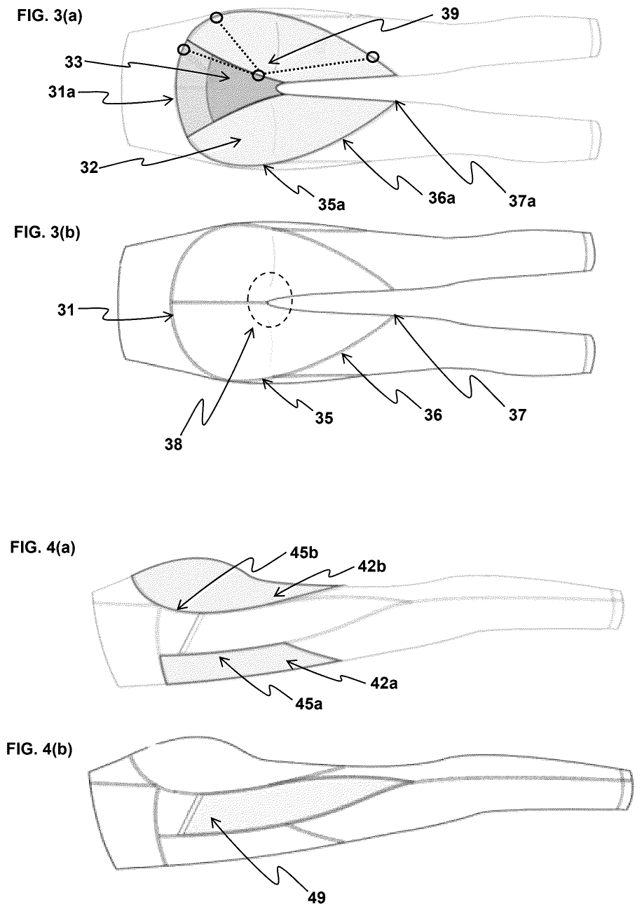

FIG. 3(b) illustrates a rear external view of one embodiment of a pant garment as described herein in the form of leggings or tights and comprising an MMFA and fabric saddle. There is a visible seam or stitching 31 close to the waistband and situated in a lower back/upper buttock region of the torso opening of the pant garment. There is also visible stitching on the outer (lateral) face of the general upper thigh area 35, spanning across the back (dorsal) face of the general lower thigh area 36 to the inner (medial) face of the general knee area 37 of the pant garment. The MMFA or any stitching or seams associated therewith is not externally visible in the crotch region as highlighted by circle 38. FIG. 3(a) illustrates a rear view of the same pant garment as FIG. 3(b) but when viewed inside out in which fabric saddle 32 has extremities that extend in length from a fabric saddle boundary at the waistband situated in a lower back/upper buttock region of the torso opening of the pant garment 31a internally into each leg opening down the outer (lateral) face of the general upper thigh area 35a, spanning across the back (dorsal) face of a general lower thigh area 36a to the inner (medial) face of the general knee area 37a of the pant garment. The MMFA 33 is visibly located in the crotch region of the pant garment in this view. A non-limiting example of suitable measurements 39 between the one point on the boundary of the crotch region and various points on the fabric saddle boundary where it is fixedly attached to the pant garment are indicated for confirming that the fabric saddle boundary is distally located from the crotch region. The difference between the product of FIG. 2 and FIG. 3 is the precise placing of the seams that hold the saddle in place in the pant garment. It will be appreciated that the position of seam (once remote from the crotch) can be selected to at least some degree for enhanced product aesthetics.

FIG. 4(b) illustrates a side on external view of one embodiment of a pant garment as described herein in the form of leggings or tights and comprising an MMFA and fabric saddle as before. In this embodiment, there is an externally visible pocket 49 included extending along the outer (lateral) face of the general upper thigh area to general knee area of the pant garment. FIG. 4(a) illustrates a side on view of the same pant garment FIG. 4(b) but when viewed inside out and in which fabric saddle 42a (ventral) and 42b (dorsal) have fabric saddle boundary 45a (ventral) and 45b (dorsal).

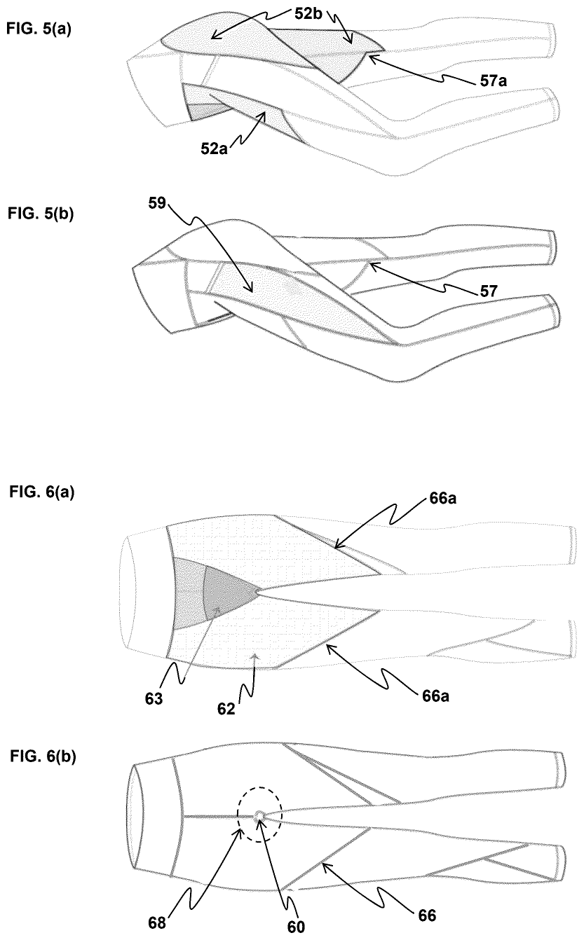

FIG. 5(b) illustrates a side on inner leg/walking view of the external view of one embodiment of a pant garment as described herein in the form of leggings or tights and comprising an MMFA and fabric saddle in which there is a visible pocket 59 included extending along the outer (lateral) face of the general upper thigh area to general knee area of the pant garment. Visible stitching 57 is shown extending to the inner (medial) face of the general knee area of the pant garment. FIG. 5(a) illustrates a side on inner leg/walking view of the same pant garment as FIG. 5(b) but when viewed inside out and in which fabric saddle 52a (ventral) and 52b (dorsal) have fabric saddle boundary 57a in the inner (medial) face of the general knee area.

FIG. 6(b) illustrates a front view of the external view of another embodiment of a pant garment as described herein in the form of leggings or tights and comprising an MMFA and fabric saddle in which an alternative visible stitching pattern 66 (to that depicted in FIG. 2) is evident spanning across the front (ventral) face of the general lower thigh area of the pant garment. A small crotch shaping panel 60—in some embodiments triangular or approximately triangular in shape—formed from the shell/outer fabric and provided for ease of fit of the pant garment is also shown. The MMFA is not visible in the crotch region as highlighted by circle 68. FIG. 6(a) illustrates a front view of the same pant garment as FIG. 6(b) but when viewed inside out in which fabric saddle 62 has an alternative fabric saddle boundary shape 66a spanning across the front (ventral) face of the general lower thigh area of the pant garment. MMFA 63 is shown located in the crotch region of the pant garment.

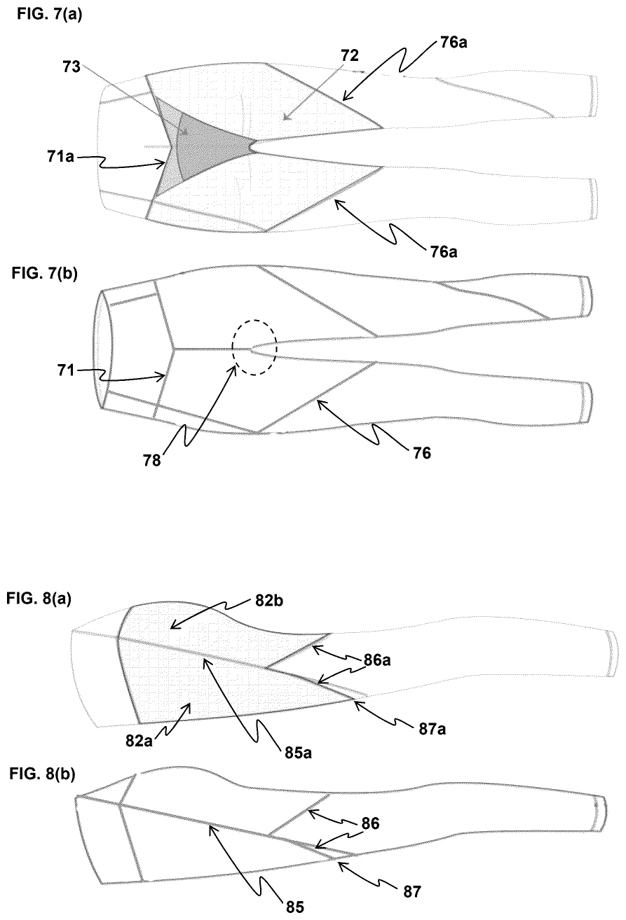

FIG. 7(b) illustrates a back external view of another embodiment of a pant garment as described herein in the form of leggings or tights and comprising an MMFA and fabric saddle in which an alternative visible stitching pattern 71 (to that depicted in FIG. 3) at a waistband situated in a lower back/upper buttock region of the torso opening of the pant garment is evident. An alternative visible stitching pattern 76 is evident spanning across the back (dorsal) face of the general lower thigh area of the pant garment. The MMFA is not visible in the crotch region as highlighted by circle 78. FIG. 7(a) illustrates a back view of the same pant garment as FIG. 7(b) but when viewed inside out in which fabric saddle 72 has an alternative fabric saddle boundary shape 76a spanning across the back (dorsal) face of the general lower thigh area of the pant garment. An alternative stitching pattern 71a is also evident at the waistband situated in the lower back/upper buttock region of the torso opening of the pant garment. MMFA 73 is shown located in the crotch region of the pant garment.

FIG. 8(b) illustrates a side on external view of another embodiment of a pant garment as described herein in the form of leggings or tights and comprising an MMFA and fabric saddle in which there is an alternative visible stitching pattern 85, 86 and 87 extending along the outer (lateral) face of the general upper thigh area (85) through to the lower thigh area (86) and finishing at the general knee area (87) of the pant garment. FIG. 8(a) illustrates a side on view of the same pant garment FIG. 8(b) but when viewed inside out and in which fabric saddle 82a (ventral) and 82b (dorsal) have fabric saddle boundary 85a (ventral) extending along the outer (lateral) face of the general upper thigh area (85a) through to the lower thigh area (86a) and finishing at the general knee area (87a) of the pant garment.

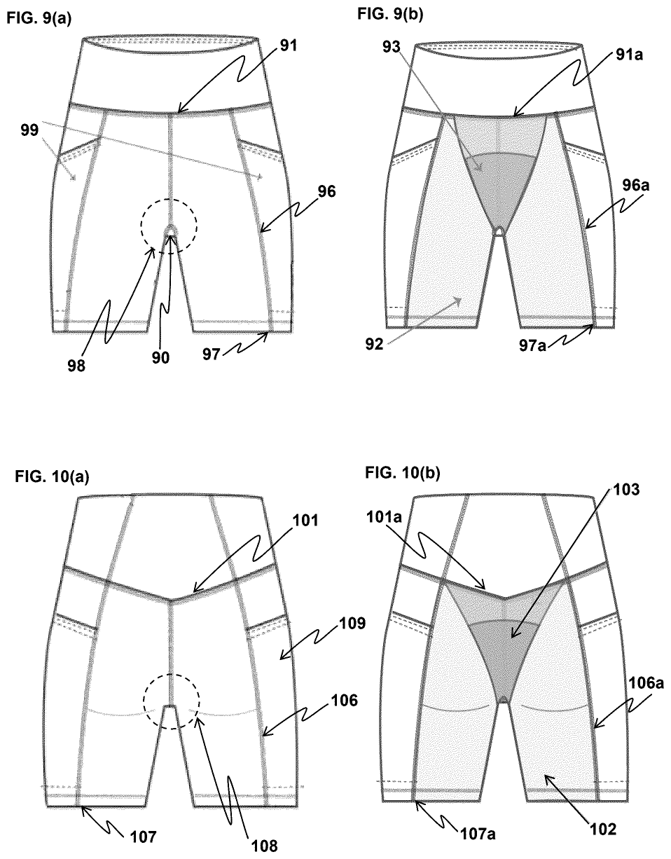

FIG. 9(a) illustrates a front external view of one embodiment of a pant garment as described herein in the form of cycle shorts and comprising an MMFA and fabric saddle in which there is visible stitching 96 extending in length from a waistband 91 partially situated in a front abdominal region of the torso opening of the pant garment along each leg opening along a front (ventral) face of the general upper and mid-thigh area of the pant garment, terminating at a distal edge 97 of the pant garment positioned circumferentially around a wearer's leg above the wearer's knee. A small crotch shaping panel 90—in some embodiments triangular or approximately triangular in shape—formed from the shell/outer fabric and provided for ease of fit of the pant garment is also shown. The MMFA is not visible in the crotch region as highlighted by circle 98. Two side pockets 99 are also visible along the outer (lateral) face of the general upper and mid-thigh area of the pant garment. FIG. 9(b) illustrates a front view of the same pant garment as FIG. 9(a) but when viewed inside out in which fabric saddle 92 has extremities 96a that extend in length from a fabric saddle boundary starting at a waistband 91a situated in a front abdominal region of the torso opening of the pant garment internally into each leg opening along the front (ventral) face of the general upper and mid-thigh area of the pant garment, terminating at a distal edge 97a of the pant garment positioned circumferentially around a wearer's leg above the wearer's knee. MMFA 93 is shown located in the crotch region of the pant garment.

FIG. 10(a) illustrates a rear external view of one embodiment of a pant garment as described herein in the form of cycle shorts and comprising an MMFA and fabric saddle in which there is visible stitching 101 at a waistband situated in a lower back/upper buttock region of the torso opening of the pant garment, visible stitching 106 along the back (dorsal) face of the general upper and mid-thigh area of the pant garment, terminating at a distal edge 107 of the pant garment positioned circumferentially around a wearer's leg above the wearer's knee. The MMFA is not visible in the crotch region as highlighted by circle 108. Two side pockets 109 are also visible along the outer (lateral) face of the general upper and mid-thigh area of the pant garment. FIG. 10(b) illustrates a rear view of the same pant garment as FIG. 10(a) but when viewed inside out in which fabric saddle 102 has extremities 106a that extend in length from a fabric saddle boundary 101a at the waistband in a lower back/upper buttock region of the torso opening of the pant garment internally into each leg opening along the back (dorsal) face of the general upper and mid-thigh area of the pant garment, terminating at a distal edge 107a of the pant garment positioned circumferentially around a wearer's leg above the wearer's knee. MMFA 103 is shown located in the crotch region of the pant garment.

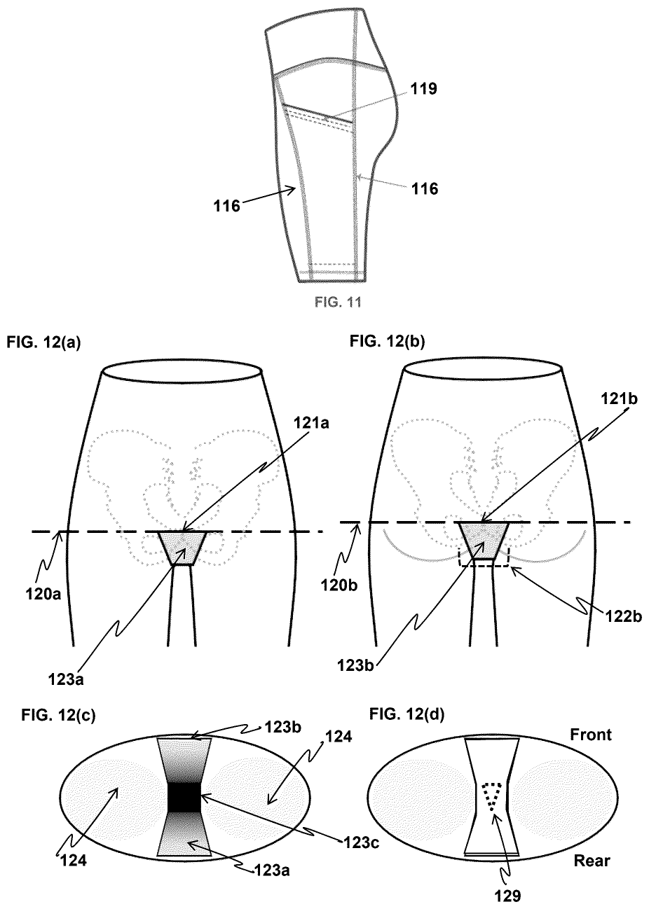

FIG. 11 illustrates a side on external view of one embodiment of a pant garment as described herein in the form of cycle shorts and comprising an MMFA and fabric saddle in which there is a visible pocket 119 included extending along the outer (lateral) face of the general upper thigh area to general knee area of the pant garment with visible stitching 116.

FIG. 12(a) illustrates a non-limiting example of a front (ventral face) view of a crotch region on a stylised human body with the approximate location of the pelvis shown in grey dotted lines. The approximate position of the top of the mons pubis, shown in black dashed line 120a, is seen overlaying the pubic symphysis 121a. The crotch region 123a is depicted extending forwardly and upwardly in an approximately trapezoidal shape from between the legs (the crotch) to cover the mons pubis. FIG. 12(b) illustrates a non-limiting example of a back (dorsal face) view of a crotch region on a stylised human body with the approximate location of the pelvis shown in grey dotted lines. The approximate position of the anus 121b is shown having vertical position on the buttocks indicated by black dashed line 120b. The crotch region 123b is depicted extending rearwardly and upwardly in an approximately trapezoidal shape from between the legs (the crotch) to the anus and extending as wide as the sit bones (the left and right ischial tuberosities) as indicated by black dashed bracket 122b. FIG. 12(c) illustrates a non-limiting example of a top view of a crotch region as viewed through the torso opening of a pant garment. Leg openings 124 are shown in grey shading positioned either side of generally rectangular crotch 123c with forward (ventrally) extending crotch region 123a having generally trapezoidal shape and rearward (dorsally) extending crotch region 123b also having a generally trapezoidal shape. This view enables visualisation of the generally flared rectangular crotch region as described in some embodiments herein, where the generally flared rectangle has a generally elongate shape and flares outwardly (i.e., away) from its longitudinal axis. FIG. 12(d) illustrates the same non-limiting example of a top view of a crotch region of a pant garment as FIG. 12(c) as viewed through the torso opening of the pant garment and shows an exemplary position and size of where a crotch shaping panel 129 may reside in the outer layer or shell of the pant garment relative to the position of the crotch region between a wearer's legs.

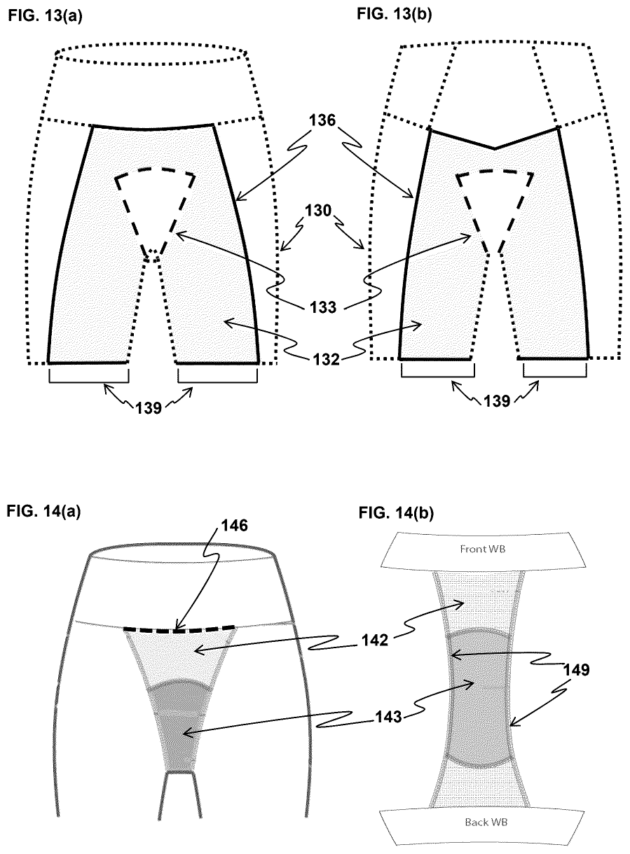

FIG. 13(a) illustrates a front internal (inside out) view and FIG. 13(b) illustrates a rear internal (inside out) view of one embodiment of a pant garment as described herein in the form of cycle shorts and comprising an MMFA 133 (shown in broken dashed lines) and a fabric saddle 132 (shaded) in which fabric saddle boundary 136 is shown as solid lines. Remaining seams/edges of the pant garment 130 are shown as dotted lines. As described herein, the fabric saddle 132 is fixedly attached along all or part of the fabric saddle boundary 136 to an interior surface of the pant garment. Seams or joins 139 along the cuffs of the pant garment may be fixedly attached to the interior surface of the pant body in some embodiments. In other embodiments, seams or joins 139 along the cuffs of the pant garment are not fixedly attached to the interior surface of the pant body.

FIG. 14(a) illustrates a front internal (inside out) view of one embodiment of a pant garment as described herein comprising an MMFA 143 and a fabric saddle 142 in which fabric saddle boundary where it is fixedly attached to an interior surface of the pant garment is shown as a single dashed line 146. FIG. 14(b) illustrates a top internal view of the same pant garment as FIG. 14(a) where MMFA 143 and fabric saddle 142 are shown attached to a front and back waistband (WB) portion of the torso opening of the pant garment, showing the fabric saddle boundary where it is not attached to an interior surface of the pant garment as seams 149.

1. A launderable moisture management outer wear pant garment for exercise comprising
a moisture management fabric assembly for managing bodily fluid secretions disposed in an internal crotch region of the pant garment so as to closely contact a wearer's skin during use;
a fabric saddle which is fixedly attached to an interior surface of the pant garment along a part or full length of an outer boundary of the fabric saddle, which outer boundary is distally located from the internal crotch region where the moisture management fabric assembly is disposed, wherein the moisture management fabric assembly is at least partially attached to the fabric saddle,
wherein the fabric saddle and the distal location of the fabric saddle boundary indirectly attaches the moisture management fabric assembly to the outer wear pant garment in a manner that avoids externally visible seams in the crotch region.

2. The garment of claim 1, wherein no portion of the moisture management fabric assembly is directly attached to the crotch region of the pant garment.

3. The garment of claim 1, wherein the fabric saddle is fixedly attached to the interior surface of the pant garment solely along a part or full length of the fabric saddle boundary and remains unattached to the interior surface of the pant garment along all other surfaces of the fabric saddle, optionally wherein the fabric saddle is fixedly attached to the interior surface of the pant garment solely along a full length of the fabric saddle boundary.

4. The garment of claim 1, wherein the moisture management fabric assembly is at least partially attached to a fabric saddle which is fixedly attached to an interior surface of the pant garment along a full length of a fabric saddle boundary which is distally located from the crotch region.

5. The garment of claim 1, wherein the fabric saddle has extremities that extend in length from at least two edges of the moisture management fabric assembly to the fabric saddle boundary.

6. The garment of claim 1, wherein the fabric saddle has extremities that extend in length to the fabric saddle boundary.

7. The garment of claim 1, wherein the moisture management fabric assembly is located over a central saddle point of the fabric saddle.

8. The garment of claim 1, wherein the fabric saddle flares upwardly towards a torso opening of the pant garment such that the fabric saddle boundary is partially situated in at least a front abdominal region and at least a lower back region of the torso opening.

9. The garment of claim 1, wherein the fabric saddle has extremities that extend in length and in opposite directions from at least two edges of the moisture management fabric assembly to the fabric saddle boundary, wherein the extremities of the fabric saddle flare upwardly towards a torso opening of the pant garment such that the fabric saddle boundary is partially situated in at least a front abdominal region and at least a lower back region of the torso opening.

10. The garment of claim 1, wherein the fabric saddle has a generally flared hyperbolic paraboloid shape comprising a central saddle point disposed in the crotch region of the pant garment.

11. The garment of claim 1, wherein the fabric saddle extends internally into each leg opening of the pant garment such that at portion of the fabric saddle boundary is partially situated in each of leg opening.

12. The garment of claim 1, wherein at least a portion of the fabric saddle boundary is located proximate to a general knee area of the pant garment and/or at least a portion of the fabric saddle boundary is located proximate to a general lower thigh area of the pant garment.

13. The garment of claim 1, wherein the fabric saddle comprises two or more fabric pieces connected together.

14. The garment of claim 1, wherein fabric of the fabric saddle comprises one or more layers of a fabric, preferably, at least one of which is a fabric mesh.

15. The garment of claim 1, wherein fabric of the fabric saddle comprises one or more layers of a natural fabric, a synthetic fabric, a semi synthetic fabric or a combination thereof.

16. The garment of claim 1, wherein fabric of the fabric saddle comprises a synthetic fabric, preferably a nylon/spandex blend.

17. The garment of claim 1, wherein the moisture management fabric assembly has opposing long edges situated between a wearer's legs proximate each leg opening of the pant garment and opposing short edges, wherein the moisture management fabric assembly is fixedly attached to the fabric saddle at least along the opposing long edges.

18. The garment of claim 1, wherein the moisture management fabric assembly comprises a fluid absorbing fabric core for trapping bodily fluid secretions.

19. The garment of claim 1, wherein the moisture management fabric assembly comprises a fluid absorbing fabric core including one or more layers of an absorbent, quick drying fabric.

20. The garment of claim 1, wherein the moisture management fabric assembly comprises a fluid wicking fabric for directly contacting a wearer's skin in use, wherein the fluid wicking fabric is positioned adjacent to a fluid absorbing fabric core.

21. The garment of claim 1, wherein the moisture management fabric assembly comprises a fluid wicking fabric selected from cotton, a cotton blend such as a cotton elastane jersey, bamboo fleece, wool such as Merino wool, or a brushed fabric having a brushed or otherwise textured surface having fibres displaced from the fabric knit, such as a brushed wool, particularly brushed Merino wool, a brushed wicking polyester, a brushed wicking polypropylene, a brushed wicking nylon, a brushed wicking micromodal, a brushed wicking bamboo, a brushed cotton, or a composite or blend thereof, most preferably brushed Merino wool.

22. The garment of claim 1, wherein the moisture management fabric assembly comprises a fluid barrier fabric to prevent fluid leaking outside the pant during use, wherein the fluid barrier fabric is positioned adjacent to the fluid absorbing fabric core, preferably wherein the fluid barrier fabric comprises a fluid proof fabric, a fluid resistant fabric or a fluid repellent fabric.

23. The garment of claim 1, wherein the externally visible seams in the crotch region exclude seams of a crotch shaping panel in an outer layer or shell of the pant garment, if present.

24. The garment of claim 1 in the form of tights, leggings or shorts, including cycle shorts.

25. The garment of claim 1, wherein the fabric saddle includes a ventral portion and a dorsal portion, and the ventral portion and the dorsal portion only connect with each other at the internal crotch region, thus providing a gap between the ventral portion and the dorsal portion along all outer thigh areas of the pant garment.

26. The garment of claim 1, wherein the moisture management fabric assembly is unattached to the fabric saddle at its short edge at a front portion of the fabric saddle.

27. The garment of claim 1, wherein the moisture management fabric saddle comprises Merino wool.
